(19) United States
(12) Patent Application Publication
(10) Pub. No.: US 2025/0129358 A1
(43) Pub. Date: Apr. 24, 2025

(54) POLYMERS, COMPOSITIONS, AND METHODS FOR ISOLATION OF NUCLEIC ACIDS

(71) Applicant: Promega Corporation, Madison, WI (US)

(72) Inventors: Zhiyang Zeng, Madison, WI (US); Andrew Taft, Madison, WI (US); Kasen Riemersma, Madison, WI (US); Spencer Hermanson, Madison, WI (US); Wenhui Zhou, Madison, WI (US)

(21) Appl. No.: 18/923,104

(22) Filed: Oct. 22, 2024

Related U.S. Application Data
(60) Provisional application No. 63/592,717, filed on Oct. 24, 2023.

(51) Int. Cl. C12N 15/10; C08F 26/06

(52) U.S. Cl. CPC C12N 15/1013; C08F 26/06

(57) ABSTRACT

Provided herein are polymeric compounds, compositions, and methods for isolation of nucleic acids from a sample.

### CROSS-REFERENCE TO RELATED APPLICATIONS

This application claims priority to and the benefit of U.S. Provisional Patent Application No. 63/592,717, filed on Oct. 24, 2023, which is incorporated herein by reference in its entirety.

### FIELD

Provided herein are polymeric compounds, compositions, and methods for isolation of nucleic acids from a sample.

### BACKGROUND

Isolation of nucleic acids from samples is a key step in many biochemical and diagnostic procedures, such as those that rely on nucleic acid amplification or sequencing reactions. Although compositions and methods for isolating nucleic acids from samples are available, many are time-consuming and/or involve use of components, such as chaotropic reagents, salts, surfactants, and organic solvents, that are incompatible with downstream applications (e.g., sequencing reactions).

### SUMMARY

In one aspect, disclosed herein is a composition comprising:
a solid surface; and
a polymer covalently or non-covalently bound to the solid surface;
wherein the polymer comprises at least one pendant moiety of formula (I):

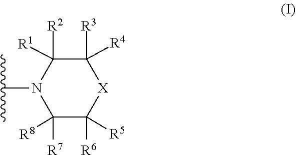

wherein:
X is selected from O, S, S(O), and S(O)₂; and
R¹, R², R³, R⁴, R⁵, R⁶, R⁷, and R⁸ are each independently selected from hydrogen, C₁-C₆ alkyl, C₂-C₆ alkenyl, C₂-C₆ alkynyl, C₁-C₆ haloalkyl, C₃-C₆ cycloalkyl, aryl, heteroaryl, heterocyclyl, halo, cyano, nitro, —OR^{a}, —SR^{b}, —C(O)OR^{c}, —C(O)NR^{d}R^{e}, —OC(O)NR^{f}R^{g}, —NR^{h}C(O)NRⁱR^{j}, and —NR^{h}C(S)NRⁱR^{j}, wherein each R^{a}, R^{h}, R^{e}, R^{d}, R^{e}, R^{f}, R^{g}, R^{h}, Rⁱ, and R^{j} is independently selected from hydrogen, C₁-C₆ alkyl, C₂-C₆ alkenyl, C₂-C₆ alkynyl, C₁-C₆ haloalkyl, C₃-C₆ cycloalkyl, and aryl.

In some embodiments, the solid surface comprises a material selected from silica, glass, a polymer, and a metal. In some embodiments, the solid surface comprises a polymer selected from cellulose, cellulose acetate, nitrocellulose, nylon, a polyester, a polyethylene, a polyethersulfone, a polyolefin, polyvinylidene fluoride, a polyacrylate, a polystyrene, or any combination thereof. In some embodiments, the solid surface is in the form of a bead, a resin, a magnetic particle, a membrane, a vial, a plate, a film, a tube, a syringe, a cartridge, a cassette, a pipette tip, a microfluidic cartridge, or a cuvette.

In some embodiments, the polymer comprises a backbone and a plurality of pendant moieties of formula (I), wherein each pendant moiety of formula (I) is bound to the backbone via a linker comprising one or more methylene, ether, ester, amide, carbamate, carbonate, urea, thioether, thioester, thioamide, thiocarbamate, thiocarbonate, thiourea, arylene, heteroarylene, or heterocyclylene moieties, or any combination thereof. In some embodiments, the linker comprises one or more —CH₂—, —O—, —C(O)O—, —C(O)NH—, —NHC(O)O—, —OC(O)O—, —NHC(O)NH—, —S—, —C(O)S—, —C(S)NH—, —NHC(S)O—, —OC(S)O—, —NHC(S)NH—, arylene, heteroarylene, or heterocyclylene moieties, or any combination thereof.

In some embodiments, the linker has a structure:

—(CH₂)ₙ₁—Z¹—(CH₂)ₙ₂—Z²—

wherein n1 and n2 are each independently selected from 1, 2, 3, 4, 5, 6, 7, and 8, and Z¹ and Z² are each independently selected from —NHC(O)NH—, —C(O)NH—, —OC(O)NH—, —OC(O)O—, —NHC(O)S—, —NHC(S)NH—, —O—, —C(O)O—, and a bond.

In some embodiments, the linker is selected from:

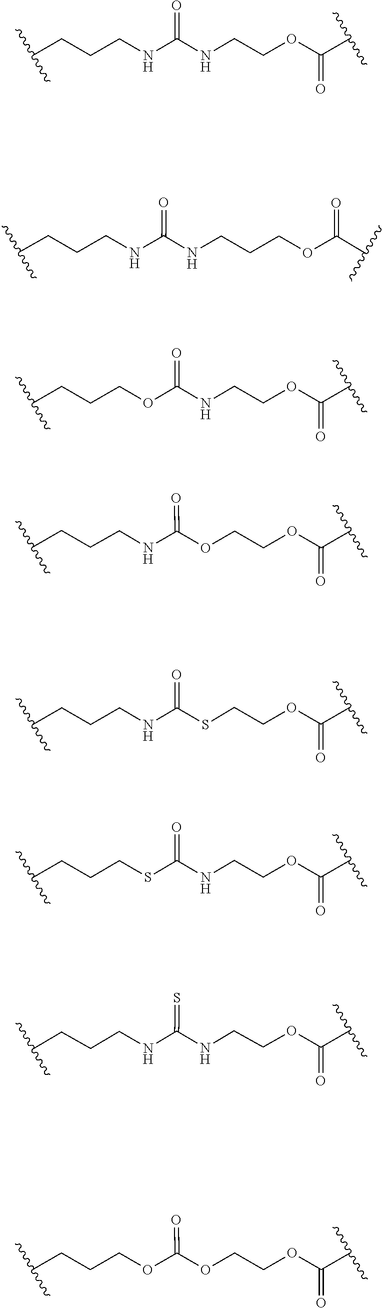

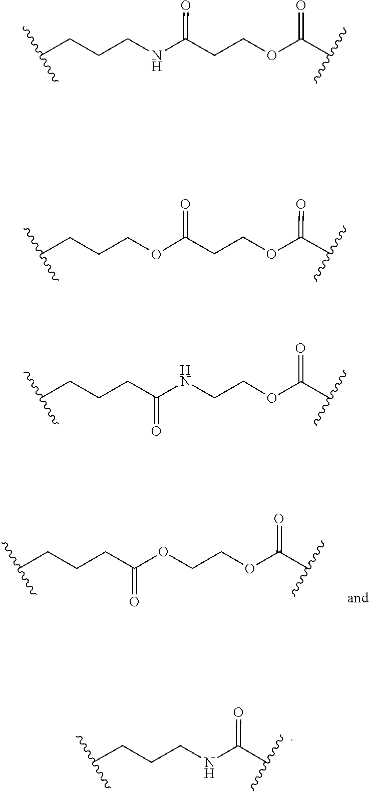 [SMILES: *CCCNC(=O)CCOC(*)=O ; *CCCOC(=O)CCOC(*)=O ; *CCCC(=O)NCCOC(*)=O ; *CCCC(=O)OCCOC(*)=O ; *CCCNC(*)=O]

In some embodiments, the linker is:

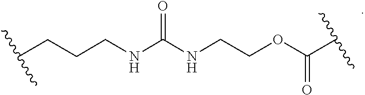 [SMILES: *CCCNC(=O)NCCOC(*)=O]

In some embodiments, X is O.

In some embodiments, R¹, R², R³, R⁴, R⁵, R⁶, R⁷, and R⁸ are each hydrogen.

In some embodiments, the polymer comprises a monomer unit of formula (Ia):

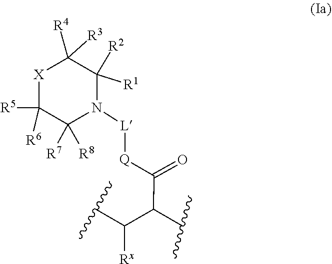

wherein:
L′ is a linker of formula: —(CH₂)ₙ₁—Z¹—(CH₂)ₙ₂—Z²—, wherein n1 and n2 are each independently selected from 1, 2, 3, 4, 5, 6, 7, and 8, and Z¹ and Z² are each independently selected from —NHC(O)NH—, —C(O)NH—, —OC(O)NH—, —OC(O)O—, —NHC(O)S—, —NHC(S)NH—, —O—, —C(O)O—, and a bond;
Q is O or NH; and
R^{x} is selected from hydrogen and methyl.

In some embodiments, the monomer unit of formula (Ia) is a monomer unit of formula:

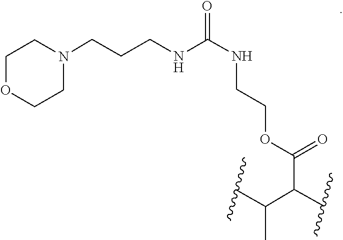

In some embodiments, the polymer further comprises one or more (meth)acrylate or (meth)acrylamide monomer units. In some embodiments, the polymer further comprises at least one (meth)acrylate or (meth)acrylamide monomer unit comprising a trialkoxysilyl group. In some embodiments, the polymer further comprises at least one monomer unit having the formula:

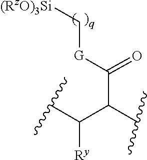

wherein: R^{y} is selected from hydrogen and methyl; each R^{z} is independently C₁-C₄ alkyl; G is O or NH; and q is 1, 2, 3, 4, 5, or 6. In some embodiments, each R^{z} is methyl, G is O, and q is 3.

In some embodiments, the polymer is covalently bound to the solid surface. In some embodiments, the polymer is non-covalently bound to the solid surface. In some embodiments, the composition further comprises a second polymer covalently or non-covalently bound to the solid surface, wherein the second polymer comprises at least one pendant moiety of formula (I).

In some embodiments, the solid surface further comprises one or more additional moieties bound to the surface (e.g., covalently bound to the surface or non-covalently bound to the surface). In some embodiments, the solid surface further comprises at least one polyethylene glycol (PEG) moiety bound to the surface.

In another aspect, disclosed herein is a method of isolating a nucleic acid from a sample, comprising:
(a) providing a sample containing a nucleic acid;
(b) contacting the sample with a composition disclosed herein (e.g., a composition disclosed herein, e.g., a composition comprising: a solid surface and a polymer covalently or non-covalently bound to the solid surface, wherein the polymer comprises at least one pendant moiety of formula (I)) at a pH of less than about 8.0, wherein the nucleic acid binds to the composition to provide a nucleic acid bound composition; and
(c) separating the nucleic acid bound composition from the sample.

In some embodiments, step (b) is conducted at a pH of about 2.0 to less than about 8.0. In some embodiments, step (b) is conducted at a pH of about 6.0 to about 7.5. In some embodiments, step (b) comprises contacting the sample with the composition for about 10 seconds to about 10 minutes. In some embodiments, step (b) comprises contacting the sample with the composition for about 1 minute to about 5 minutes.

In some embodiments, the method further comprises one or more steps of washing the nucleic acid bound composition after step (c) by contacting the composition with a wash solution having a pH of less than about 8.0.

In some embodiments, the method further comprises a step of contacting the nucleic acid bound composition with an elution solution at a pH of about 8.5 or higher, to release the nucleic acid from the composition. In some embodiments, the elution solution comprises an aqueous buffer. In some embodiments, the method further comprises contacting the nucleic acid bound composition with the elution solution at a pH of about 8.5 to about 9.5. In some embodiments, the method further comprises contacting the nucleic acid bound composition with the elution solution at a pH of about 8.5 to about 9.0. In some embodiments, the method further comprises contacting the nucleic acid bound composition with the elution solution for about 10 seconds to about 10 minutes. In some embodiments, the method further comprises contacting the nucleic acid bound composition with the elution solution for about 1 minute to about 5 minutes.

In some embodiments, the method further comprises performing a polymerase chain reaction on the nucleic acid bound composition.

In some embodiments, the sample comprises a body fluid. In some embodiments, the body fluid is selected from blood, saliva, lymph, milk, mucus, urine, sweat, amniotic fluid, cerebrospinal fluid, feces, a vaginal secretion, and semen. In some embodiments, the sample comprises cultured cells, tissue cells, or cells from a body fluid (e.g., blood, saliva, lymph, milk, mucus, urine, sweat, amniotic fluid, cerebrospinal fluid, feces, a vaginal secretion, or semen). In some embodiments, the sample is a blood sample selected from whole blood, plasma, and serum. In some embodiments, the sample is an environmental sample selected from soil and water. In some embodiments, the sample is a food sample (e.g., a meat sample). In some embodiments, the sample is a plant (e.g., a vegetable). In some embodiments, the sample comprises a virus, bacteria, a fungus (e.g., a yeast or a mold), or any combination thereof. In some embodiments, the sample comprises media from cultured cells, a cell lysate, a cell supernatant, or a purified fraction of a cell (e.g., a subcellular fraction).

In some embodiments, the method comprises a step of lysing the sample prior to step (b).

In some embodiments, the nucleic acid is DNA. In some embodiments, the DNA is selected from total DNA, mtDNA (mitochondrial DNA), gDNA (genomic DNA), cfDNA (cell-free DNA), ccfDNA (circulating cell-free DNA), cffDNA (cell free fetal DNA), bacterial DNA, viral DNA, and ctDNA (circulating tumor DNA). In some embodiments, the nucleic acid is RNA. In some embodiments, the RNA is selected from total RNA, miRNA, mRNA, tRNA, rRNA, siRNA, ctRNA (circulating tumor RNA), and viral RNA. In some embodiments, the nucleic acid is total nucleic acids from the sample.

Also disclosed herein is a method of isolating a nucleic acid from a sample, comprising:
(a) providing a sample containing a nucleic acid;
(b) contacting the sample with a polymer at a pH of less than about 8.0, wherein the nucleic acid binds to the polymer to provide a nucleic acid bound polymer; and
(c) separating the nucleic acid bound polymer from the sample, wherein the polymer comprises at least one pendant moiety of formula (I):

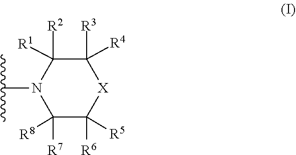

wherein:
X is selected from O, S, S(O), and S(O)₂; and
R¹, R², R³, R⁴, R⁵, R⁶, R⁷, and R⁸ are each independently selected from hydrogen, C₁-C₆ alkyl, C₂-C₆ alkenyl, C₂-C₆ alkynyl, C₁-C₆ haloalkyl, C₃-C₆ cycloalkyl, aryl, heteroaryl, heterocyclyl, halo, cyano, nitro, —OR^{a}, —SR^{b}, —C(O)OR^{c}, —C(O)NR^{d}R^{e}, —OC(O)NR^{f}R^{g}, —NR^{h}C(O)NRⁱR^{j}, and —NR^{h}C(S)NRⁱR^{j}, wherein each R^{a}, R^{h}, R^{e}, R^{d}, R^{e}, R^{f}, R^{g}, R^{h}, Rⁱ, and R^{j} is independently selected from hydrogen, C₁-C₆ alkyl, C₂-C₆ alkenyl, C₂-C₆ alkynyl, C₁-C₆ haloalkyl, C₃-C₆ cycloalkyl, and aryl.

In some embodiments, the polymer comprises a backbone and a plurality of pendant moieties of formula (I), wherein each pendant moiety of formula (I) is bound to the backbone via a linker comprising one or more methylene, ether, ester, amide, carbamate, carbonate, urea, thioether, thioester, thioamide, thiocarbamate, thiocarbonate, thiourea, arylene, heteroarylene, or heterocyclylene moieties, or any combination thereof. In some embodiments, the linker comprises one or more —CH₂—, —O—, —C(O)O—, —C(O)NH—, —NHC(O)O—, —OC(O)O—, —NHC(O)NH—, —S—, —C(O)S—, —C(S)NH—, —NHC(S)O—, —OC(S)O—, —NHC(S)NH—, arylene, heteroarylene, or heterocyclylene moieties, or any combination thereof.

In some embodiments, the linker has a structure:

—(CH₂)ₙ₁—Z¹—(CH₂)ₙ₂—Z²—

wherein n1 and n2 are each independently selected from 1, 2, 3, 4, 5, 6, 7, and 8, and Z¹ and Z² are each independently selected from —NHC(O)NH—, —C(O)NH—, —OC(O)NH—, —OC(O)O—, —NHC(O)S—, —NHC(S)NH—, —O—, —C(O)O—, and a bond.

In some embodiments, the linker is selected from:

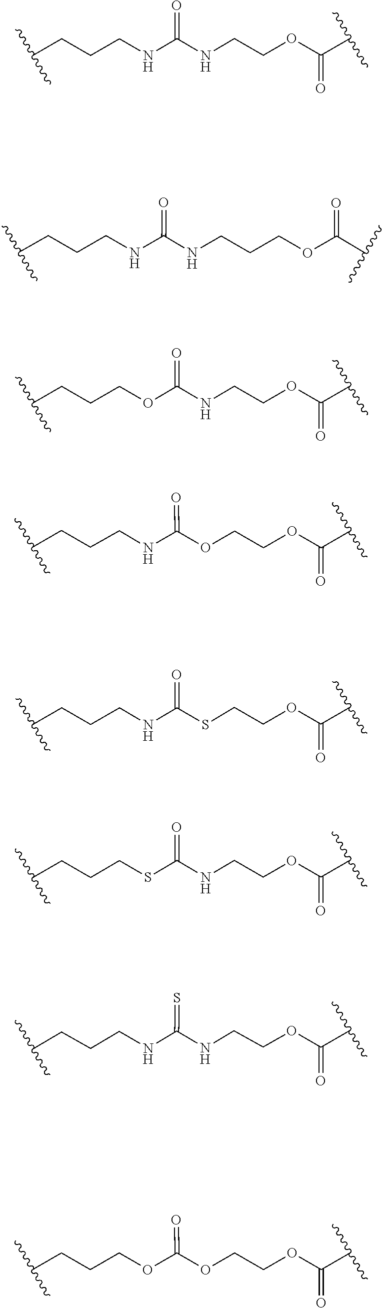

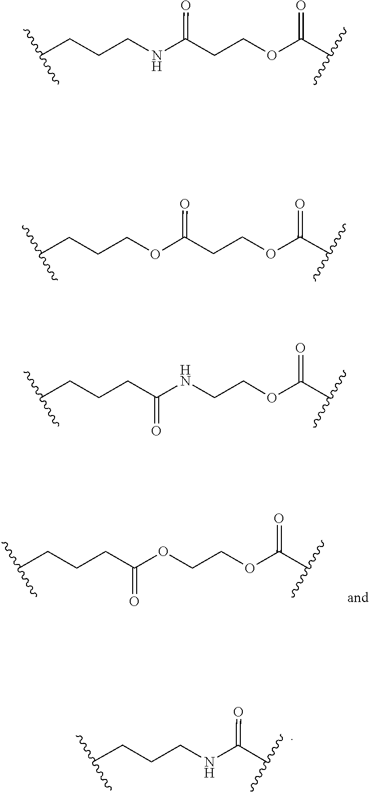 [SMILES: *CCCNC(=O)CCOC(*)=O ; *CCCOC(=O)CCOC(*)=O ; *CCCC(=O)NCCOC(*)=O ; *CCCC(=O)OCCOC(*)=O ; *CCCNC(*)=O]

In some embodiments, the linker is:

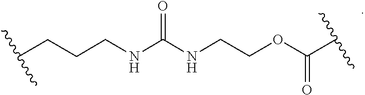 [SMILES: *CCCNC(=O)NCCOC(*)=O]

In some embodiments, X is O.

In some embodiments, R¹, R², R³, R⁴, R⁵, R⁶, R⁷, and R⁸ are each hydrogen.

In some embodiments, the polymer comprises a monomer unit of formula (Ia):

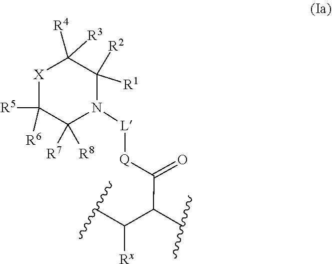

wherein:
L′ is a linker of formula: —(CH₂)ₙ₁—Z¹—(CH₂)ₙ₂—Z²—, wherein n1 and n2 are each independently selected from 1, 2, 3, 4, 5, 6, 7, and 8, and Z¹ and Z² are each independently selected from —NHC(O)NH—, —C(O)NH—, —OC(O)NH—, —OC(O)O—, —NHC(O)S—, —NHC(S)NH—, —O—, —C(O)O—, and a bond;
Q is O or NH; and
R^{x} is selected from hydrogen and methyl.

In some embodiments, the monomer unit of formula (Ia) is a monomer unit of formula:

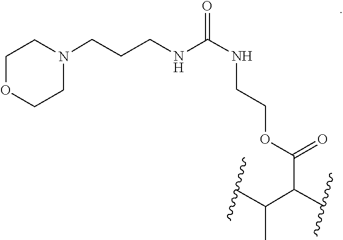

In some embodiments, the polymer further comprises one or more (meth)acrylate monomer units. In some embodiments, the polymer further comprises at least one (meth)acrylate monomer unit comprising a trialkoxysilyl group. In some embodiments, the polymer further comprises at least one monomer unit having the formula:

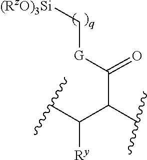

wherein R^{y} is selected from hydrogen and methyl, each R^{z} is independently C₁-C₄ alkyl, G is O or NH, and q is 1, 2, 3, 4, 5, or 6. In some embodiments, each R^{z} is methyl, G is O, and q is 3.

In some embodiments, step (b) is conducted at a pH of about 2.0 to less than about 8.0. In some embodiments, step (b) is conducted at a pH of about 6.0 to about 7.5. In some embodiments, step (b) comprises contacting the sample with the composition for about 10 seconds to about 10 minutes. In some embodiments, step (b) comprises contacting the sample with the composition for about 1 minute to about 5 minutes.

In some embodiments, the method further comprises one or more steps of washing the nucleic acid bound composition after step (c) by contacting the composition with a wash solution having a pH of less than about 8.0.

In some embodiments, the method further comprises a step of contacting the nucleic acid bound composition with an elution solution at a pH of about 8.5 or higher, to release the nucleic acid from the composition. In some embodiments, the elution solution comprises an aqueous buffer. In some embodiments, the method comprises contacting the nucleic acid bound composition with the elution solution at a pH of about 8.5 to about 9.5. In some embodiments, the method comprises contacting the nucleic acid bound composition with the elution solution at a pH of about 8.5 to about 9.0. In some embodiments, the method comprises contacting the nucleic acid bound composition with the elution solution for about 10 seconds to about 10 minutes. In some embodiments, the method comprises contacting the nucleic acid bound composition with the elution solution for about 1 minute to about 5 minutes.

In some embodiments, the method further comprises performing a polymerase chain reaction on the nucleic acid bound composition.

In some embodiments, the sample comprises a body fluid. In some embodiments, the body fluid is selected from blood, saliva, lymph, milk, mucus, urine, sweat, amniotic fluid, cerebrospinal fluid, feces, a vaginal secretion, and semen. In some embodiments, the sample comprises cultured cells, tissue cells, or cells from a body fluid (e.g., blood, saliva, lymph, milk, mucus, urine, sweat, amniotic fluid, cerebrospinal fluid, feces, a vaginal secretion, or semen). In some embodiments, the sample is a blood sample selected from whole blood, plasma, and serum. In some embodiments, the sample is an environmental sample selected from soil and water. In some embodiments, the sample is a food sample (e.g., a meat sample). In some embodiments, the sample is a plant (e.g., a vegetable). In some embodiments, the sample comprises a virus, bacteria, a fungus (e.g., a yeast or a mold), or any combination thereof. In some embodiments, the sample comprises media from cultured cells, a cell lysate, a cell supernatant, or a purified fraction of a cell (e.g., a subcellular fraction).

In some embodiments, the method comprises a step of lysing the sample prior to step (b).

In some embodiments, the nucleic acid is DNA. In some embodiments, the DNA is selected from total DNA, mtDNA (mitochondrial DNA), gDNA (genomic DNA), cfDNA (cell-free DNA), ccfDNA (circulating cell-free DNA), cffDNA (cell free fetal DNA), bacterial DNA, viral DNA, and ctDNA (circulating tumor DNA). In some embodiments, the nucleic acid is RNA. In some embodiments, the RNA is selected from total RNA, miRNA, mRNA, tRNA, rRNA, siRNA, ctRNA (circulating tumor RNA), and viral RNA. In some embodiments, the nucleic acid is total nucleic acids from the sample.

Also disclosed herein is a copolymer comprising:
(a) a plurality of monomer units of formula (Ia):

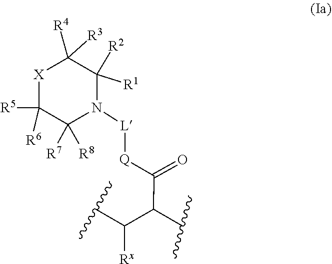

wherein:
X is selected from O, S, S(O), and S(O)₂;
R¹, R², R³, R⁴, R⁵, R⁶, R⁷, and R⁸ are each independently selected from hydrogen, C₁-C₆ alkyl, C₂-C₆ alkenyl, C₂-C₆ alkynyl, C₁-C₆ haloalkyl, C₃-C₆ cycloalkyl, aryl, heteroaryl, heterocyclyl, halo, cyano, nitro, —OR^{a}, —SR^{b}, —C(O)OR^{c}, —C(O)NR^{d}R^{e}, —OC(O)NR^{f}R^{g}, NR^{h}C(O)NRⁱR^{j}, and —NR^{h}C(S)NRⁱR^{j} wherein each R^{a}, R^{b}, R^{e}, R^{d}, R^{c}, R^{f}, R^{g}, R^{h}, Rⁱ, and R^{j} is independently selected from hydrogen, C₁-C₆ alkyl, C₂-C₆ alkenyl, C₂-C₆ alkynyl, C₁-C₆ haloalkyl, C₃-C₆ cycloalkyl, and aryl;
L′ is a linker of formula: —(CH₂)ₙ₁—Z¹—(CH₂)ₙ₂—Z²—, wherein n1 and n2 are each independently selected from 1, 2, 3, 4, 5, 6, 7, and 8, and Z¹ and Z² are each independently selected from —NHC(O)NH—, —C(O)NH—, —OC(O)NH—, —OC(O)O—, —NHC(O)S—, —NHC(S)NH—, —O—, —C(O)O—, and a bond;
Q is O or NH; and
R^{x} is selected from hydrogen and methyl; and
(b) a plurality of monomer units of formula (Ib):

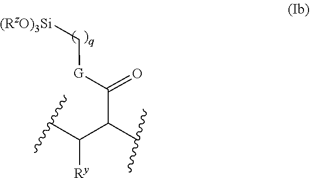

wherein:
R^{y} is selected from hydrogen and methyl;
each R^{z} is independently C₁-C₄ alkyl;
G is O or NH; and
q is 1, 2, 3, 4, 5, or 6.

### BRIEF DESCRIPTION OF THE DRAWINGS

FIGS. 1A-1B show illustrations of polymers disclosed herein bound to solid surfaces (e.g., magnetic silica beads). FIG. 1A shows an example of a polymer covalently bound to a solid surface. FIG. 1B shows an example of a polymer non-covalently bound to a solid surface.

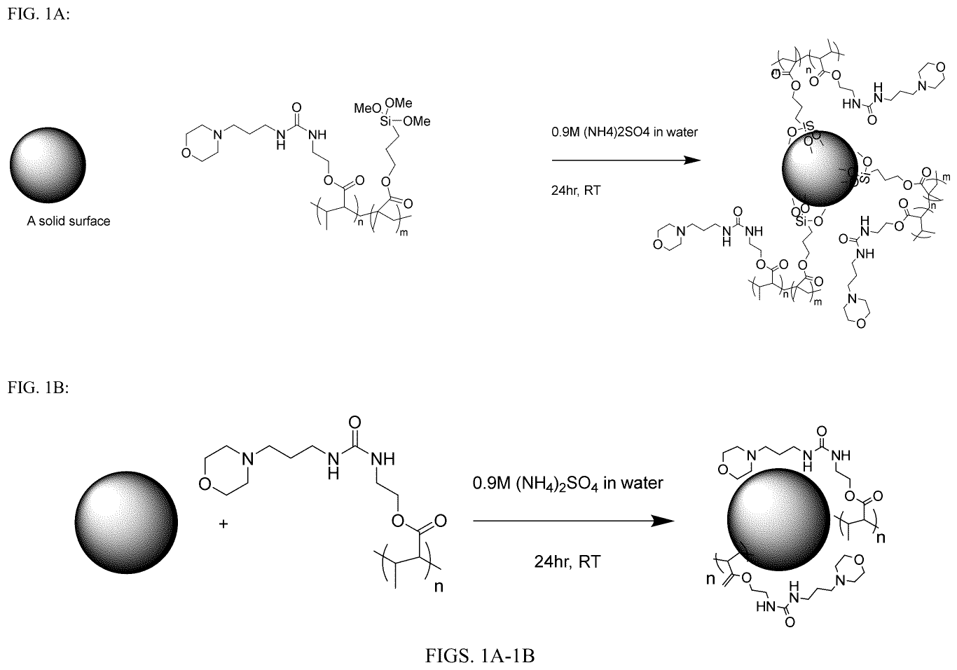

FIG. 2 shows DNA binding percentage and elution percentage from polymer-bound magnetic silica particles.

FIG. 3 shows isolation and concentration of ccfDNA (circulating cell-free DNA) using polymer-bound magnetic silica particles.

### DETAILED DESCRIPTION

Provided herein are polymeric compounds, compositions, and methods for isolation of nucleic acids from a sample. The polymeric compounds and compositions allow for capture of nucleic acids at low pH (e.g., less than about 8). Upon a shift to a higher pH (e.g., about 8.5 or above), nucleic acids are released into solution. The use of polymers allows for a high density of the functional groups (e.g., morpholino groups) that bind to the nucleic acids. In some embodiments, the entire process can be conducted in less than 10 minutes, and the nucleic acids can be released under conditions that are compatible with downstream applications such as polymerase chain reaction (PCR), including reverse transcription PCR (RT-PCR).

### I. Definitions

Although any methods and materials similar or equivalent to those described herein can be used in the practice or testing of embodiments described herein, some preferred methods, compositions, devices, and materials are described herein. However, before the present materials and methods are described, it is to be understood that this disclosure is not limited to the particular molecules, compositions, methodologies, or protocols as herein described as these may vary in accordance with routine experimentation and optimization. It is also to be understood that the terminology used in the description is for the purpose of describing the particular versions or embodiments only and is not intended to limit the scope of the embodiments described herein.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics, and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein and in the appended claims, the singular forms “a,” “an,” and “the” include plural reference unless the context clearly dictates otherwise. Thus, for example, reference to “a peptide” is a reference to one or more peptides and equivalents thereof known to those skilled in the art, and so forth.

As used herein, the term “and/or” includes any and all combinations of listed items, including any of the listed items individually. For example, “A, B, and/or C” encompasses A, B, C, AB, AC, BC, and ABC, each of which is to be considered separately described by the statement “A, B, and/or C.”

As used herein, the term “comprise” and linguistic variations thereof denote the presence of recited feature(s), element(s), method step(s), etc., without the exclusion of the presence of additional feature(s), element(s), method step(s), etc. Conversely, the term “consisting of” and linguistic variations thereof, denotes the presence of recited feature(s), element(s), method step(s), etc., and excludes any unrecited feature(s), element(s), method step(s), etc., except for ordinarily-associated impurities. The phrase “consisting essentially of” denotes the recited feature(s), element(s), method step(s), etc., and any additional feature(s), element(s), method step(s), etc., that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Sorrell, Organic Chemistry, 2^{nd} edition, University Science Books, Sausalito, 2006; Smith, March's Advanced Organic Chemistry: Reactions, Mechanism, and Structure, 7^{th} Edition, John Wiley & Sons, Inc., New York, 2013; Larock, Comprehensive Organic Transformations, 3^{rd} Edition, John Wiley & Sons, Inc., New York, 2018; and Carruthers, Some Modern Methods of Organic Synthesis, 3^{rd} Edition, Cambridge University Press, Cambridge, 1987; the entire contents of each of which are incorporated herein by reference.

As used herein, the term “alkyl” refers to a straight or branched saturated hydrocarbon chain. The alkyl chain can include, e.g., from 1 to 30 carbon atoms (C₁-C₃₀ alkyl), 1 to 24 carbon atoms (C₁-C₂₄ alkyl), for example 1 to 16 carbon atoms (C₁-C₁₆ alkyl), 1 to 14 carbon atoms (C₁-C₁₄ alkyl), 1 to 12 carbon atoms (C₁-C₁₂ alkyl), 1 to 10 carbon atoms (C₁-C₁₀ alkyl), 1 to 8 carbon atoms (C₁-C₈ alkyl), 1 to 6 carbon atoms (C₁-C₆ alkyl), or 1 to 4 carbon atoms (C₁-C₄ alkyl). Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, n-undecyl, and n-dodecyl.

As used herein, the term “alkenyl” refers to a straight or branched hydrocarbon chain containing at least one carbon-carbon double bond. The double bond(s) may be located at any position with the hydrocarbon chain. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

As used herein, the term “alkynyl” refers to a straight or branched hydrocarbon chain containing at least one carbon-carbon triple bond. The triple bond(s) may be located at any position with the hydrocarbon chain. Representative examples of alkynyl include, but are not limited to, ethynyl, propynyl, and butynyl.

As used herein, the term “aryl” refers to an aromatic carbocyclic ring system having a single ring (monocyclic) or multiple rings (bicyclic or tricyclic) including fused ring systems, and zero heteroatoms. As used herein, aryl contains 6-20 carbon atoms (C₆-C₂₀ aryl), 6 to 14 ring carbon atoms (C₆-C₁₄ aryl), 6 to 12 ring carbon atoms (C₆-C₁₂ aryl), or 6 to 10 ring carbon atoms (C₆-C₁₀ aryl). Representative examples of aryl groups include, but are not limited to, phenyl, naphthyl, anthracenyl, and phenanthrenyl.

As used herein, the term “cyano” refers to a group —CN.

As used herein, the term “cycloalkyl” refers to a saturated carbocyclic ring system containing three to ten carbon atoms and zero heteroatoms. The cycloalkyl may be monocyclic, bicyclic, bridged, fused, or spirocyclic. Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, bicyclo[2.2.1]heptanyl, bicyclo[3.2.1]octanyl, and bicyclo[5.2.0]nonanyl.

As used herein, the term “halogen” or “halo” means F, Cl, Br, or I.

As used herein, the term “haloalkyl” refers to an alkyl group, as defined herein, in which at least one hydrogen atom (e.g., one, two, three, four, five, six, seven or eight hydrogen atoms) is replaced with a halogen. In some embodiments, each hydrogen atom of the alkyl group is replaced with a halogen. Representative examples of haloalkyl include, but are not limited to, fluoromethyl, difluoromethyl, trifluoromethyl, 2,2,2-trifluoroethyl, and 3,3,3-trifluoropropyl.

As used herein, the term “heteroaryl” refers to an aromatic group having a single ring (monocyclic) or multiple rings (bicyclic or tricyclic) having one or more ring heteroatoms independently selected from O, N, and S. The aromatic monocyclic rings are five- or six-membered rings containing at least one heteroatom independently selected from O, N, and S (e.g., 1, 2, 3, or 4 heteroatoms independently selected from O, N, and S). The five-membered aromatic monocyclic rings have two double bonds, and the six-membered aromatic monocyclic rings have three double bonds. The bicyclic heteroaryl groups are exemplified by a monocyclic heteroaryl ring appended fused to a monocyclic aryl group, as defined herein, or a monocyclic heteroaryl group, as defined herein. The tricyclic heteroaryl groups are exemplified by a monocyclic heteroaryl ring fused to two rings independently selected from a monocyclic aryl group, as defined herein, and a monocyclic heteroaryl group as defined herein. Representative examples of monocyclic heteroaryl include, but are not limited to, pyridinyl (including pyridin-2-yl, pyridin-3-yl, pyridin-4-yl), pyrimidinyl, pyrazinyl, pyridazinyl, pyrrolyl, benzopyrazolyl, 1,2,3-triazolyl, 1,3,4-thiadiazolyl, 1,2,4-thiadiazolyl, 1,3,4-oxadiazolyl, 1,2,4-oxadiazolyl, imidazolyl, thiazolyl, isothiazolyl, thienyl, furanyl, oxazolyl, isoxazolyl, 1,2,4-triazinyl, and 1,3,5-triazinyl. Representative examples of bicyclic heteroaryl include, but are not limited to, benzimidazolyl, benzodioxolyl, benzofuranyl, benzooxadiazolyl, benzopyrazolyl, benzothiazolyl, benzothienyl, benzotriazolyl, benzoxadiazolyl, benzoxazolyl, chromenyl, imidazopyridine, imidazothiazolyl, indazolyl, indolyl, isobenzofuranyl, isoindolyl, isoquinolinyl, naphthyridinyl, purinyl, pyridoimidazolyl, quinazolinyl, quinolinyl, quinoxalinyl, thiazolopyridinyl, thiazolopyrimidinyl, thienopyrrolyl, and thienothienyl. Representative examples of tricyclic heteroaryl include, but are not limited to, dibenzofuranyl and dibenzothienyl. The monocyclic, bicyclic, and tricyclic heteroaryls are connected to the parent molecular moiety through any carbon atom or any nitrogen atom contained within the rings.

As used herein, the term “heterocycle” or “heterocyclic” refers to a saturated or partially unsaturated non-aromatic cyclic group having one or more ring heteroatoms independently selected from O, N, and S. means a monocyclic heterocycle, a bicyclic heterocycle, or a tricyclic heterocycle. The monocyclic heterocycle is a three-, four-, five-, six-, seven-, or eight-membered ring containing at least one heteroatom independently selected from O, N, and S. The three- or four-membered ring contains zero or one double bond, and one heteroatom selected from O, N, and S. The five-membered ring contains zero or one double bond and one, two or three heteroatoms selected from O, N, and S. The six-membered ring contains zero, one, or two double bonds and one, two, or three heteroatoms selected from O, N, and S. The seven- and eight-membered rings contains zero, one, two, or three double bonds and one, two, or three heteroatoms selected from O, N, and S. Representative examples of monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, oxetanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a phenyl group, or a monocyclic heterocycle fused to a monocyclic cycloalkyl, or a monocyclic heterocycle fused to a monocyclic cycloalkenyl, or a monocyclic heterocycle fused to a monocyclic heterocycle, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. Representative examples of bicyclic heterocycles include, but are not limited to, benzopyranyl, benzothiopyranyl, chromanyl, 2,3-dihydrobenzofuranyl, 2,3-dihydrobenzothienyl, 2,3-dihydroisoquinoline, 2-azaspiro[3.3]heptan-2-yl, azabicyclo[2.2.1]heptyl (including 2-azabicyclo[2.2.1]hept-2-yl), 2,3-dihydro-1H-indolyl, isoindolinyl, octahydrocyclopenta[c]pyrrolyl, octahydropyrrolopyridinyl, and tetrahydroisoquinolinyl. Tricyclic heterocycles are exemplified by a bicyclic heterocycle fused to a phenyl group, or a bicyclic heterocycle fused to a monocyclic cycloalkyl, or a bicyclic heterocycle fused to a monocyclic cycloalkenyl, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan,hexahydro-1H-1,4-methanocyclopenta[c]furan, aza-adamantane (1-azatricyclo[3.3.1.1^{3,7}]decane), and oxa-adamantane (2-oxatricyclo[3.3.1.1^{3,7}]decane). The monocyclic, bicyclic, and tricyclic heterocycles are connected to the parent molecular moiety through any carbon atom or any nitrogen atom contained within the rings.

As used herein, the term “(meth)” designates optional methyl substitution. Thus, a term such as “(meth)acrylates” denotes both methacrylates and acrylates.

As used herein, the term “nitro” refers to a group —NO₂.

As used herein, the term “sample” is used in its broadest sense. In one sense, it is meant to include a specimen or culture obtained from any source as well as biological and environmental samples. Biological samples may be obtained from animals (including humans) and encompass fluids, solids, tissues, and gases. Biological samples include blood products such as plasma, serum, and the like. Sample may also refer to cells or cell lysates. Cell lysates may include cells that have been lysed with a lysing agent or lysates such as rabbit reticulocyte or wheat germ lysates. Environmental samples include environmental material such as surface matter, soil, water (e.g., wastewater), crystals, and industrial samples. Sample may also include purified samples, such as purified protein samples. Such examples are not however to be construed as limiting the sample types applicable to the present disclosure.

When a group or moiety can be substituted, the term “substituted” indicates that one or more (e.g., 1, 2, 3, 4, 5, or 6; in some embodiments 1, 2, or 3; and in other embodiments 1 or 2) hydrogens on the group indicated in the expression using “substituted” can be replaced with a selection of recited indicated groups or with a suitable substituent group known to those of skill in the art (e.g., one or more of the groups recited below), provided that the designated atom's normal valence is not exceeded. Substituent groups include, but are not limited to, alkyl, alkenyl, alkynyl, alkoxy, acyl, amino, amido, amidino, aryl, azido, carbamoyl, carboxyl, carboxyl ester, cyano, cycloalkyl, cycloalkenyl, guanidino, halo, haloalkyl, haloalkoxy, heteroalkyl, heteroaryl, heterocyclyl, hydroxy, hydrazino, imino, oxo, nitro, phosphate, phosphonate, sulfonic acid, thiol, thione, or combinations thereof.

As used herein, in chemical structures the indication:

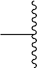

represents a point of attachment of one moiety to another moiety (e.g., a substituent group to the rest of the compound).

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

Where substituent groups are specified by their conventional chemical formulae, written from left to right, they optionally encompass substituents resulting from writing the structure from right to left, e.g., —CH₂O— optionally also recites —OCH₂—, and —OC(O)NH— also optionally recites —NHC(O)O—.

### II. Compositions and Compounds

Provided herein are polymeric compounds and compositions that can be used to isolate nucleic acids from samples. The polymeric compounds and compositions include a morpholino moiety or a derivative thereof. At lower pH (e.g., less than about 8), the morpholine moieties are protonated and positively charged, and thus can bind negatively charged nucleic acids. Upon a shift to higher pH (e.g., about 8.5 or higher), the morpholine moieties are deprotonated such that the nucleic acids are released into solution. The disclosed compositions include polymers having morpholino moieties, or derivatives thereof (e.g., morpholine, thiomorpholine, thiomorpholine 1-oxide, thiomorpholine 1,1-dioxide, or similar materials salts thereof), which are covalently or non-covalently bound to the polymer backbone. The morpholino moieties, or derivatives thereof, can be incorporated into the polymers by inclusion of a monomer that includes the morpholino moiety (or derivative thereof), or by first preparing a polymer and subsequently attaching a morpholino moiety (or derivative thereof), e.g., by reacting the polymer with a compound having a morpholino moiety (or derivative thereof) and a reactive group.

The polymers and compositions including morpholine groups (or derivatives thereof) can release high quantities of the nucleic acids (e.g., in some embodiments, at least 80% of bound nucleic acids can be released in less than 5 minutes, or less than 1 minute). This provides a significant advantage to the disclosed materials. Furthermore, the nucleic acids can be released using elution solutions that do not require components (e.g., chaotropic agents) that can disrupt downstream applications, such as sequencing reactions. Accordingly, the disclosed polymeric compounds and compositions can be used for efficient isolation of nucleic acids to provide a solution that can be used directly in downstream applications without further sample processing.

Accordingly, disclosed herein are polymers comprising at least one pendant moiety of formula (I):

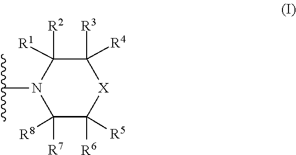

wherein:
X is selected from O, S, S(O), and S(O)₂;
R¹, R², R³, R⁴, R⁵, R⁶, R⁷, and R⁸ are each independently selected from hydrogen, C₁-C₆ alkyl, C₂-C₆ alkenyl, C₂-C₆ alkynyl, C₁-C₆ haloalkyl, C₃-C₆ cycloalkyl, aryl, heteroaryl, heterocyclyl, halo, cyano, nitro, —OR^{a}, —SR^{b}, —C(O)OR^{c}, —C(O)NR^{d}R^{e}, —OC(O)NR^{f}R^{g}, NR^{h}C(O)NRⁱR^{j}, and —NR^{h}C(S)NRⁱR^{j} wherein each R^{a}, R^{b}, R^{e}, R^{d}, R^{e}, R^{f}, R^{g}, R^{h}, R¹, and R^{j} is independently selected from hydrogen, C₁-C₆ alkyl, C₂-C₆ alkenyl, C₂-C₆ alkynyl, C₁-C₆ haloalkyl, C₃-C₆ cycloalkyl, and aryl.

Also disclosed herein are compositions comprising: a solid surface; and a polymer covalently or non-covalently bound to the solid surface, wherein the polymer comprises at least one pendant moiety of formula (I) as defined above.

In the pendant moiety of formula (I), X is selected from O, S, S(O), and S(O)₂. In some embodiments, X is O. In some embodiments, X is S. In some embodiments, X is S(O). In some embodiments, X is S(O)₂.

In the pendant moiety of formula (I), R¹, R², R³, R⁴, R⁵, R⁶, R⁷, and R⁸ are each independently selected from hydrogen, C₁-C₆ alkyl, C₂-C₆ alkenyl, C₂-C₆ alkynyl, C₁-C₆ haloalkyl, C₃-C₆ cycloalkyl, aryl, heteroaryl, heterocyclyl, halo, cyano, nitro, —OR^{a}, —SR^{b}, —C(O)OR^{c}, —C(O)NR^{d}R^{e}, —OC(O)NR^{f}R^{g}, —NR^{h}C(O)NRⁱR^{j}, and —NR^{h}C(S)NRⁱR^{j}, wherein each R^{a}, R^{b}, R^{e}, R^{d}, R^{e}, R^{f}, R^{g}, R^{h}, Rⁱ, and R^{j} is independently selected from hydrogen, C₁-C₆ alkyl, C₂-C₆ alkenyl, C₂-C₆ alkynyl, C₁-C₆ haloalkyl, C₃-C₆ cycloalkyl, and aryl. In some embodiments, R¹, R², R³, R⁴, R⁵, R⁶, R⁷, and R⁸ are each independently selected from hydrogen, C₁-C₃ alkyl, halo, and —OR^{a}, wherein R^{a} is selected from hydrogen and C₁-C₃ alkyl. In some embodiments, one or two of R¹, R², R³, R⁴, R⁵, R⁶, R⁷, and R⁸ are each independently selected from hydrogen, C₁-C₆ alkyl, C₂-C₆ alkenyl, C₂-C₆ alkynyl, C₁-C₆ haloalkyl, C₃-C₆ cycloalkyl, aryl, heteroaryl, heterocyclyl, halo, cyano, nitro, —OR^{a}, —SR^{b}, —C(O)OR^{c}, —C(O)NR^{d}R^{e}, —OC(O)NR^{f}R^{g}, —NR^{h}C(O)NRⁱR^{j}, and —NR^{h}C(S)NRⁱR^{j}, and the rest are hydrogen. In some embodiments, one or two of R¹, R², R³, R⁴, R⁵, R⁶, R⁷, and R⁸ are each independently selected from hydrogen, C₁-C₃ alkyl, halo, and —OR^{a}, wherein R^{a} is selected from hydrogen and C₁-C₃ alkyl, and the rest are hydrogen. In some embodiments, R¹, R², R³, R⁴, R⁵, R⁶, R⁷, and R⁸ are each hydrogen.

The polymer comprising the pendant moiety of formula (I) comprises a backbone, to which the pendant moiety of formula (I) is bound via a linker. In some embodiments, the linker is a direct bond. In other embodiments, the linker separates the moiety of formula (I) from the polymer backbone by about 5 Å, about 10 Å, about 20 Å, about 50 Å, about 100 Å, about 150 Å, about 200 Å, about 300 Å, about 400 Å, about 500 Å, about 600 Å, about 700 Å, about 800 Å, about 900 Å, about 1000 Å, or any suitable range therebetween (e.g., about 5-100 Å, about 50-500 Å, about 150-700 Å, etc.). In some embodiments, the linker separates the moiety of formula (I) from the polymer backbone by about 1-200 atoms (e.g., about 1, about 2, about 3, about 4, about 5, about 6, about 7, about 8, about 9, about 10, about 15, about 20, about 25, about 30, about 35, about 40, about 45, about 50, about 60, about 70, about 80, about 90, about 100, about 120, about 140, about 160, about 180, about 200, or any suitable ranges therebetween (e.g., about 2-20, about 10-50, etc.)).

The linker can include one or more groups independently selected from methylene (—CH₂—), ethylene (—CH═CH—), ethynylene (—C≡C—), ether (—O—), amine (—NR—, wherein R is hydrogen or an alkyl group), thioether (—S—), carbonyl (—C(O)—), thiocarbonyl (—C(S)—), sulfonyl (—S(O)₂—), arylene, heteroarylene, and heterocyclylene moieties, or any combination thereof. For example, the above moieties can be combined to form additional groups that may be included in the linker, e.g., a carbonyl group and an ether group can together provide an ester moiety (—C(O)O—); a carbonyl group and two ether groups can together provide a carbonate moiety (—OC(O)O—); a carbonyl group and an unsubstituted amine group can together provide an unsubstituted amide moiety (—C(O)NH—); a carbonyl group and two unsubstituted amine groups can together provide an unsubstituted urea moiety (—NHC(O)NH—); a carbonyl group together with an unsubstituted amine group and an ester group can provide an unsubstituted carbamate moiety (—OC(O)NH—); a carbonyl group together with a thioether and an unsubstituted amine group can provide an S-thiocarbamate moiety; a thiocarbonyl group together with an ether and an unsubstituted amine group can provide an O-thiocarbamate moiety; multiple methylene groups can together form an alkylene chain; etc.

In some embodiments, the linker comprises one or more methylene, ether, ester, amide, carbamate, carbonate, urea, thioether, thioester, thioamide, thiocarbamate, thiocarbonate, thiourea, arylene, heteroarylene, or heterocyclylene moieties, or any combination thereof. In some embodiments, the linker comprises one or more —CH₂—, —O—, —C(O)O—, —C(O)NH—, —NHC(O)O—, —OC(O)O—, —NHC(O)NH—, —S—, —C(O)S—, —C(S)NH—, —NHC(S)O—, —OC(S)O—, —NHC(S)NH—, arylene, heteroarylene, or heterocyclylene moieties, or any combination thereof.

In some embodiments, the linker comprises one or more alkylene groups (e.g., —(CH₂)ₙ—, wherein n is 1-12, e.g., 1, 2, 3, 4, 5, 6, 7, 8, 9, 10, 11, or 12, or any suitable range therebetween). In some embodiments, the linker comprises one or more branched alkylene groups.

In some embodiments, the linker has a formula —(CH₂)ₙ₁—Z¹—(CH₂)ₙ₂—Z²—, wherein n1 and n2 are each independently selected from 1, 2, 3, 4, 5, 6, 7, and 8, and Z¹ and Z² are each independently selected from —NHC(O)NH—, —C(O)NH—, —OC(O)NH—, —OC(O)O—, —NHC(O)S—, —NHC(S)NH—, —O—, —C(O)O—, and a bond. In some embodiments, the linker has a formula —(CH₂)ₙ₁—NH—C(O)—NH—(CH₂)ₙ₂—O—C(O)—, wherein n1 and n2 are each independently selected from 1, 2, 3, 4, 5, 6, 7, and 8.

In some embodiments, the linker has a formula selected from:

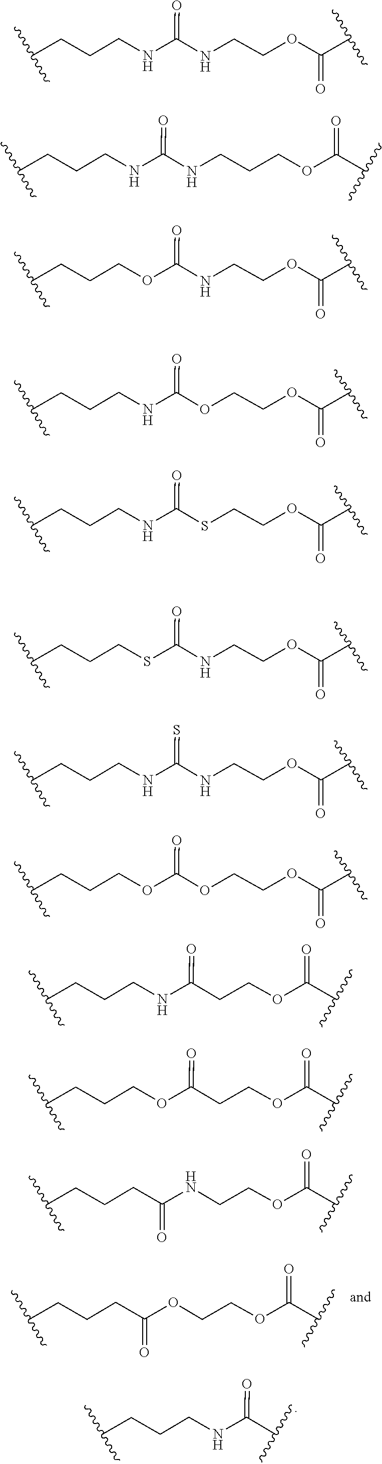

In some embodiments, the linker has a formula:

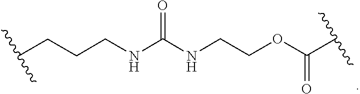 [SMILES: *CCCNC(=O)NCCOC(*)=O]

In some embodiments, the linker can include one or more moieties that results from the reaction of two reactive groups, such as reactive groups known in various bioconjugation reactions. Such moieties would result, for example, if a pendant moiety of formula (I) is installed onto a pre-existing polymer backbone with pendant complementary reactive groups. For example, the linker can include a triazole group, which is formed by the reaction of an azide and an alkyne. Exemplary moieties that may result from such reactions include, but are not limited to:

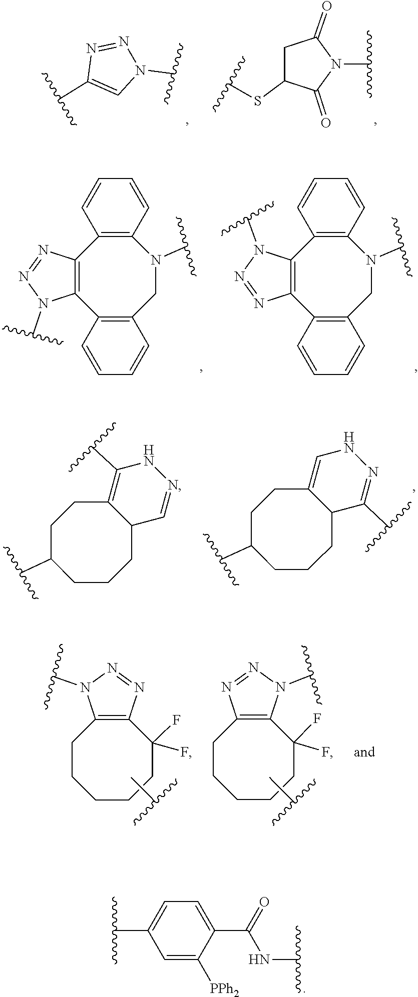

In some embodiments, the polymer comprises a monomer unit of formula (Ia):

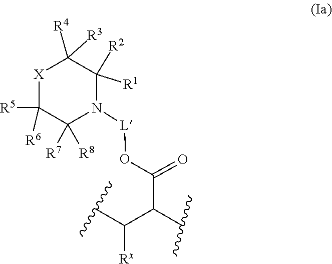

wherein:
X is selected from O, S, S(O), and S(O)₂;
R¹, R², R³, R⁴, R⁵, R⁶, R⁷, and R⁸ are each independently selected from hydrogen, C₁-C₆ alkyl, C₂-C₆ alkenyl, C₂-C₆ alkynyl, C₁-C₆ haloalkyl, C₃-C₆ cycloalkyl, aryl, heteroaryl, heterocyclyl, halo, cyano, nitro, —OR^{a}, —SR^{b}, —C(O)OR^{c}, —C(O)NR^{d}R^{e}, —OC(O)NR^{f}R^{g}, —NR^{h}C(O)NRⁱR^{j}, and —NR^{h}C(S)NRⁱR^{j}, wherein each R^{a}, R^{h}, R^{e}, R^{d}, R^{e}, R^{f}, R^{g}, R^{h}, Rⁱ, and R^{j} is independently selected from hydrogen, C₁-C₆ alkyl, C₂-C₆ alkenyl, C₂-C₆ alkynyl, C₁-C₆ haloalkyl, C₃-C₆ cycloalkyl, and aryl;
L′ is a linker of formula: —(CH₂)ₙ₁—Z¹—(CH₂)ₙ₂—Z²—, wherein n1 and n2 are each independently selected from 1, 2, 3, 4, 5, 6, 7, and 8, and Z¹ and Z² are each independently selected from —NHC(O)NH—, —C(O)NH—, —OC(O)NH—, —OC(O)O—, —NHC(O)S—, —NHC(S)NH—, —O—, —C(O)O—, and a bond;
Q is O or NH; and
R^{x} is selected from hydrogen and methyl.

In the monomer unit of formula (Ia), X is selected from O, S, S(O), and S(O)₂. In some embodiments, X is O. In some embodiments, X is S. In some embodiments, X is S(O). In some embodiments, X is S(O)₂.

In the monomer unit of formula (Ia), R¹, R², R³, R⁴, R⁵, R⁶, R⁷, and R⁸ are each independently selected from hydrogen, C₁-C₆ alkyl, C₂-C₆ alkenyl, C₂-C₆ alkynyl, C₁-C₆ haloalkyl, C₃-C₆ cycloalkyl, aryl, heteroaryl, heterocyclyl, halo, cyano, nitro, —OR^{a}, —SR^{b}, —C(O)OR^{c}, —C(O)NR^{d}R^{e}, —OC(O)NRⁱR^{j}, —NR^{h}C(O)NRⁱR^{j}, and —NR^{h}C(S)NRⁱR^{j}, wherein each R^{a}, R^{b}, R^{e}, R^{d}, R^{e}, R^{f}, R^{g}, R^{h}, Rⁱ, and R³ is independently selected from hydrogen, C₁-C₆ alkyl, C₂-C₆ alkenyl, C₂-C₆ alkynyl, C₁-C₆ haloalkyl, C₃-C₆ cycloalkyl, and aryl. In some embodiments, R¹, R², R³, R⁴, R⁵, R⁶, R⁷, and R⁸ are each independently selected from hydrogen, C₁-C₃ alkyl, halo, and —OR^{a}, wherein R^{a} is selected from hydrogen and C₁-C₃ alkyl. In some embodiments, one or two of R¹, R², R³, R⁴, R⁵, R⁶, R⁷, and R⁸ are each independently selected from hydrogen, C₁-C₆ alkyl, C₂-C₆ alkenyl, C₂-C₆ alkynyl, C₁-C₆ haloalkyl, C₃-C₆ cycloalkyl, aryl, heteroaryl, heterocyclyl, halo, cyano, nitro, —OR^{a}, —SR^{b}, —C(O)OR^{c}, —C(O)NR^{d}R^{e}, —OC(O)NRⁱR^{j}, —NR^{h}C(O)NRⁱR^{j}, and —NR^{h}C(S)NRⁱR^{j}, and the rest are hydrogen. In some embodiments, one or two of R¹, R², R³, R⁴, R⁵, R⁶, R⁷, and R⁸ are each independently selected from hydrogen, C₁-C₃ alkyl, halo, and —OR^{a}, wherein R^{a} is selected from hydrogen and C₁-C₃ alkyl, and the rest are hydrogen. In some embodiments, R¹, R², R³, R⁴, R⁵, R⁶, R⁷, and R⁸ are each hydrogen.

In the monomer unit of formula (Ia), in some embodiments, L¹ has a formula —(CH₂)ₙ₁—NH—C(O)—NH—(CH₂)ₙ₂—O—C(O)—, wherein n1 and n2 are each independently selected from 1, 2, 3, 4, 5, 6, 7, and 8.

In the monomer unit of formula (Ia), in some embodiments, Q is 0. In some embodiments, Q is NH.

In some embodiments, the monomer unit of formula (Ia) is a monomer unit of formula:

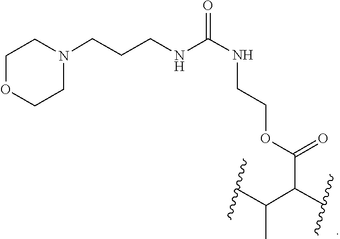

In some embodiments, the polymer further comprises one or more (meth)acrylate or (meth)acrylamide monomer units, derived from one or more (meth)acrylate or (meth)acrylamide monomers. For example, in some embodiments, the polymer further comprises at least one (meth)acrylate monomer selected from an alkyl (meth)acrylate, a hydroxyalkyl (meth)acrylate, an alkoxyalkyl (meth)acrylate, a cycloalkyl (meth)acrylate, and an aromatic (meth)acrylate.

In some embodiments, the polymer further comprises an alkyl (meth)acrylate monomer selected from methyl (meth)acrylate, ethyl (meth)acrylate, n-propyl (meth)acrylate, n-butyl (meth)acrylate, iso-butyl (meth)acrylate, tert-butyl (meth)acrylate, n-hexyl (meth)acrylate, 2-ethylhexyl (meth)acrylate, octyl (meth)acrylate, iso-decyl (meth)acrylate, heptadecyl (meth)acrylate, dodecyl (meth)acrylate, 2-propylheptyl (meth)acrylate, and stearyl (meth)acrylate.

In some embodiments, the polymer further comprises a hydroxyalkyl (meth)acrylate monomer selected from hydroxymethyl (meth)acrylate, 2-hydroxyethyl (meth)acrylate, 3-hydroxypropyl (meth)acrylate, and 4-hydroxybutyl (meth)acrylate.

In some embodiments, the polymer further comprises an alkoxyalkyl (meth)acrylate selected from 2-methoxyethyl (meth)acrylate, 2-ethoxyethyl (meth)acrylate, 1-methyl-2-methoxyethyl (meth)acrylate, ethylene glycol methyl ether (meth)acrylate, diethylene glycol methyl ether (meth)acrylate, and triethylene glycol methyl ether (meth)acrylate.

In some embodiments, the polymer further comprises a cycloalkyl (meth)acrylate selected from cyclopentyl (meth)acrylate, cyclohexyl (meth)acrylate, 2-cyclohexylethyl (meth)acrylate, 3-cyclohexylpropyl (meth)acrylate, 2-norbornyl (meth)acrylate, and isobornyl (meth)acrylate.

In some embodiments, the polymer further comprises at least two different (meth)acrylate monomers, in addition to the monomer unit having the pendant moiety of formula (I).

In some embodiments, the polymer further comprises one or more (meth)acrylamide monomer units, derived from one or more (meth)acrylamide monomers. In some embodiments, the one or more (meth)acrylamide monomers are selected from (meth)acrylamide, N-methyl(meth)acrylamide, N-ethyl(meth)acrylamide, N-isopropyl(meth)acrylamide, N,N-dimethyl(meth)acrylamide, N,N-diethyl(meth)acrylamide, N-(2-hydroxyethyl)(meth)acrylamide, N,N-bis(2-hydroxyethyl)(meth)acrylamide, N-(2-hydroxypropyl)(meth)acrylamide, N,N-bis(2-hydroxypropyl)(meth)acrylamide, N-(3-hydroxypropyl)(meth)acrylamide, N-(2-hydroxybutyl)(meth)acrylamide, N-(3-hydroxybutyl)(meth)acrylamide, N-(4-hydroxybutyl)(meth)acrylamide, N-2-aminoethyl (meth)acrylamide, N-3-aminopropyl (meth)acrylamide, N-2-aminopropyl(meth)acrylamide, N,N-bis-2-aminoethyl(meth)acrylamide, N,N-bis-3-aminopropyl (meth)acrylamide, N,N-bis-2-aminopropyl (meth)acrylamide, or the like.

In some embodiments, the polymer further comprises one or more additional monomer units, such as monomer units based on styrene, ethylene, propylene, esters, saccharides, peptides, and the like. For example, the polymer can be a polystyrene, a polyethylene, a polypropylene, a polyester, a polysaccharide, a polypeptide, or any combination thereof.

In some embodiments, the polymer further comprises at least one (meth)acrylate monomer unit comprising a trialkoxysilyl group. Such groups can be used, for example, for covalent binding of the polymer to the surface, such as a glass surface. For example, in some embodiments, the polymer further comprises at least one monomer unit having formula:

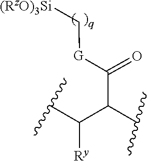

wherein: R^{y} is selected from hydrogen and methyl; each R^{z} is independently C₁-C₄ alkyl; G is O or NH; and q is 1, 2, 3, 4, 5, or 6. In some embodiments, each R^{z} is methyl, and q is 3. In some embodiments, each R^{z} is methyl, G is O, and q is 3. In some embodiments, R^{y} is methyl, each R^{z} is methyl, G is O, and q is 3.

In some embodiments, the polymer is covalently bound to the solid surface. In such embodiments, the polymer includes a moiety that is capable of covalent binding to a surface, e.g., the polymer includes a pendant group comprising a trialkoxysilyl group, which can covalently bind to a glass surface. In other embodiments, the polymer is non-covalently bound to the surface, e.g., by being adsorbed onto the surface.

In some embodiments, the composition further comprises a second polymer covalently or non-covalently bound to the solid surface, wherein the second polymer comprises at least one pendant moiety of formula (I). For example, in some embodiments, the composition further comprises a second polymer that includes the same pendant moieties of formula (I) as the first polymer, attached to a different polymer backbone. In some embodiments, the composition further comprises a second polymer that includes the same polymer backbone as the first polymer, with different pendant moieties of formula (I). The composition can include additional polymers as well.

The polymers can be prepared by standard polymerization processes, such as radical polymerization methods. Such methods include bulk, solution, suspension, and emulsion polymerization methods, and controlled radical polymerization methods such as stable free radical polymerization, nitroxide-mediated living polymerization, atom transfer radical polymerization, reversible addition fragmentation chain transfer polymerization, organotellurium mediated living radical polymerization, and the like. Such methods involve use of a polymerization initiator, which is a compound that can decompose into radicals, which subsequently react with monomer compounds to initiate a radical polymerization reaction. For example, photoinitiators decompose by photochemical processes. Typical examples of photoinitiators include, but are not limited to: benzil, benzoin, acetophenone, benzophenone, camphorquinone, or derivatives thereof; monoacyl and bisacyl phosphine oxides; and α-ketoesters including α-ketoglutaric acid, ethyl pyruvate, and the like. Combinations of photoinitiators can also be used.

For the embodiments in which the polymer is used with a solid surface, any solid surface suitable for isolating nucleic acids can be used. For example, in some embodiments, the solid surface comprises a material selected from silica, glass, a polymer, and a metal. In some embodiments, the solid surface is a glass surface. In some embodiments, the solid surface comprises silica. In some embodiments, the solid surface comprises a polymer, such as a polymer selected from cellulose, cellulose acetate, nitrocellulose, nylon, a polyester, a polyethylene, a polyethersulfone, a polyolefin, polyvinylidene fluoride, a polyacrylate, a polystyrene, or any combination thereof. The solid surface can be in the form of, for example, a bead, a resin, a magnetic particle, a membrane, a vial, a plate, a film, a tube, a syringe, a cartridge, a cassette, a pipette tip, a microfluidic cartridge, or a cuvette. In some embodiments, the solid surface is in the form of a bead. In some embodiments, the solid surface is in the form of a resin. In some embodiments, the solid surface is in the form of a membrane. In some embodiments, the solid surface is in the form of a plate. In some embodiments, the solid surface is in the form of a pipette tip. In some embodiments, the solid surface is in the form of a microfluidic cartridge.

In some embodiments, the solid surface comprises one or more additional moieties, in addition to the moiety of formula (I). For example, the solid surface may be functionalized with additional ligands. In some embodiments, the solid surface further comprises one or more PEG ligands.

### III. Methods of Use

The compositions disclosed herein (i.e., a polymer disclosed herein, or a composition comprising a polymer and a solid surface) can be used to isolate nucleic acids (e.g., DNA and RNA) from samples, e.g., biological samples. In one aspect, the disclosure provides a method of isolating a nucleic acid from a sample, comprising:
(a) providing a sample containing a nucleic acid;
(b) contacting the sample with a composition disclosed herein (e.g., a composition comprising: a solid surface; and a polymer covalently or non-covalently bound to the solid surface, wherein the polymer comprises at least one pendant moiety of formula (I)) at a pH of less than about 8.0, wherein the nucleic acid binds to the composition to provide a nucleic acid bound composition; and
(c) separating the nucleic acid bound composition from the sample.

In some embodiments, the nucleic acids isolated from the sample are selected from DNA and RNA. In some embodiments, the nucleic acids isolated from the sample are DNA (e.g., genomic DNA (gDNA), plasmid DNA, cDNA, mitochondrial DNA (mtDNA), cosmid DNA, cell-free DNA (cfDNA), circulating cell-free DNA (ccfDNA), cell-free fetal DNA (cffDNA), bacterial DNA, viral DNA, circulating tumor DNA (ctDNA), total DNA, or the like). In some embodiments, the nucleic acids isolated from the sample are RNA, e.g., messenger RNA (mRNA), microRNA (miRNA), a pre-miRNA, ribosomal RNA (rRNA), mitochondrial RNA, non-coding RNA, circular RNA, small interfering RNA (siRNA), guide RNA, total RNA, or the like. In some embodiments, the nucleic acids isolated from the sample are total nucleic acids.

Any suitable samples containing nucleic acids can be used in the disclosed methods. In some embodiments, the sample comprises a body fluid, such as blood, saliva, lymph, milk, mucus, urine, sweat, amniotic fluid, cerebrospinal fluid, feces, a vaginal secretion, or semen. In some embodiments, the sample comprises cultured cells, tissue (e.g., fresh tissue or formalin-fixed paraffin embedded (FFPE) tissue), or cells from a body fluid. In some embodiments, the sample comprises a virus, bacteria, a fungus (e.g., yeast or mold), or any combination thereof. In some embodiments, the sample is a food sample, e.g., a meat sample. In some embodiments, the sample is a plant, e.g., a vegetable. In particular embodiments, the sample is a blood sample selected from whole blood, plasma, and serum. In some embodiments, the sample is an environmental sample, such as soil or water (e.g., wastewater). The methods can also be used to isolate a nucleic acid from other environments, such as an agarose or polyacrylamide gel, or a solution in which a target nucleic acid amplification has already been carried out.

In some embodiments, the method may further comprise a step of lysing the sample prior to contacting the sample with a composition disclosed herein. Any suitable method of lysing sample can be used, including osmotic lysis (i.e., using a hypotonic solution), enzymatic lysis (e.g., using lysozyme), chemical lysis (e.g., use of detergents such as non-ionic detergents or cationic detergents, with or without other components such as sodium hydroxide), physical lysis (e.g., by mechanical lysis such as blending, shaking with glass beads, use of a homogenizer or French press, use of a sonicator, freeze-thaw cycles, or mechanical grinding), or heat lysis, or any combination thereof.

The methods include a step of contacting the sample (e.g., a lysed sample) with a polymer or composition disclosed herein (e.g., a polymer disclosed herein, or composition comprising polymer disclosed herein bound to a solid surface), such that the nucleic acids in the sample bind to the composition to provide a nucleic acid bound composition. During this contacting step, the pH if the sample is less than about 8.0, to ensure that the moiety of formula (I) in the composition is positively charged. In some embodiments, the contacting step is conducted at a pH of about 2.0 to about 8.0, about 2.5 to about 8.0, about 3.0 to about 8.0, about 3.5 to about 8.0, about 4.0 to about 8.0, about 4.5 to about 8.0, about 5.5 to about 8.0, or a pH of about 6.0 to about 7.5, e.g., a pH of about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, or any range therebetween.

In some embodiments, nucleic acids bind rapidly to the polymers or compositions disclosed herein. For example, in some embodiments, greater than about 50% of the nucleic acids in the sample (e.g., greater than about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%) bind to the polymer or composition within less than about 10 minutes (e.g., less than about 10 minutes, less than about 9 minutes, less than about 8 minutes, less than about 7 minutes, less than about 6 minutes, less than about 5 minutes, less than about 4 minutes, less than about 3 minutes, less than about 2 minutes, or less than about 1 minute). In some embodiments, greater than 75% of the nucleic acids in the sample (e.g., greater than 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%) bind to the polymer or composition within less than about 10 minutes (e.g., less than about 10 minutes, less than about 9 minutes, less than about 8 minutes, less than about 7 minutes, less than about 6 minutes, less than about 5 minutes, less than about 4 minutes, less than about 3 minutes, less than about 2 minutes, or less than about 1 minute). In some embodiments, greater than 75% of the nucleic acids in the sample (e.g., greater than 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%) bind to the polymer or composition within less than about 5 minutes (e.g. less than about 5 minutes, less than about 4 minutes, less than about 3 minutes, less than about 2 minutes, or less than about 1 minute). In some embodiments, greater than 75% of the nucleic acids in the sample (e.g., greater than 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%) bind to the polymer or composition within less than about 1 minute. Accordingly, although the step of contacting the sample with the polymer or composition can be conducted for longer periods of time if convenient or desired, in some embodiments, the contacting step is carried out for less than about 10 minutes (e.g., less than about 10 minutes, less than about 9 minutes, less than about 8 minutes, less than about 7 minutes, less than about 6 minutes, less than about 5 minutes, less than about 4 minutes, less than about 3 minutes, less than about 2 minutes, or less than about 1 minute), as this time period is sufficient to allow for binding of the nucleic acids to the polymer or composition. In some embodiments, the contacting step is carried out for about 10 seconds to about 10 minutes, or about 1 minute to about 5 minutes. The contacting step results in production of a nucleic acid bound polymer or composition.

The methods optionally include one or more washing steps after the contacting step. I some embodiments, the methods further comprise contacting at least one wash solution with the nucleic acid bound polymer or the nucleic acid bound composition. The wash solution is typically an aqueous solution (e.g., an aqueous buffer) optionally containing chemical additives, such as detergents, salts (e.g., NaCl), or an organic solvent (e.g., methanol, ethanol, isopropanol, acetonitrile, DMSO, or DMF), that has a pH of about 2.0 to about 8.0, about 2.5 to about 8.0, about 3.0 to about 8.0, about 3.5 to about 8.0, about 4.0 to about 8.0, about 4.5 to about 8.0, about 5.5 to about 8.0, or about 6.0 to about 7.5, such that the polymer or composition will remain protonated and the nucleic acid will remain bound, or aqueous while other contaminants will be washed away. In some embodiments, the wash solution is an aqueous buffer solution having a pH of about 2.0 to about 8.0, about 2.5 to about 8.0, about 3.0 to about 8.0, about 3.5 to about 8.0, about 4.0 to about 8.0, about 4.5 to about 8.0, about 5.5 to about 8.0, or about 6.0 to about 7.5, e.g., a pH of about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, or any range therebetween.

After the contacting step and the optional washing step(s), the nucleic acids bound polymer or composition can be used in a number of different applications. In some embodiments, the nucleic acids are released from the nucleic acid bound polymer or composition, e.g., by contacting the nucleic acid bound polymer or composition with an elution solution having a pH of about 8.5 or higher, e.g., a pH of about 8.5 to about 9.5, e.g., a pH of about 8.5, about 8.6, about 8.7, about 8.8, about 8.9, about 9.0, about 9.1, about 9.2, about 9.3, about 9.4, about 9.4, or about 9.5, or any range therebetween. In some embodiments, the elution solution comprises a buffer, such as tris(hydroxymethyl)aminomethane (Tris), 4-(2-hydroxyethyl)-1-piperazineethanesulfonic acid (HEPES), 3-(N-morpholino)propane sulfonic acid (MOPS), or the like. The elution solution can include other components, such as salts, so long as those components are included in concentrations that will not interfere with downstream applications such as PCR. For example, in some embodiments, the elution solution comprises a salt (e.g., NaCl) in a concentration of less than about 25 nM.

A significant advantage of the disclosed polymer or composition is that it can release nucleic acids at a very rapid rate upon a shift to a higher pH. Although other polymers or compositions are capable of binding and releasing nucleic acids, including polymers compositions similar to those described herein that have a different pendant moiety (e.g., an imidazole, a methylpiperazine, an amine (e.g., a primary amine (—NH₂) or a substituted amine (e.g., —N(CH₃)₂ or —N(CH₂CH₂OH)₂)), the polymers and compositions disclosed herein can release significant quantities of nucleic acids in a very short time frame. In some embodiments, contacting the nucleic acid bound polymer or composition with an elution solution having a pH of about 8.5 or higher results in release of more than 50% of the bound nucleic acids (e.g., greater than 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%) in less than about 5 minutes (e.g., less than about less than about 5 minutes, less than about 4 minutes, less than about 3 minutes, less than about 2 minutes, or less than about 1 minute). In some embodiments, contacting the nucleic acid bound polymer or composition with an elution solution having a pH of about 8.5 or higher results in release of more than 75% of the bound nucleic acids (e.g., greater than 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%) in less than about 5 minutes (e.g., less than about less than about 5 minutes, less than about 4 minutes, less than about 3 minutes, less than about 2 minutes, or less than about 1 minute). This greatly reduces the processing time needed to isolate nucleic acids from samples. Accordingly, although the step of contacting the nucleic acid bound polymer or composition with the elution solution can be conducted for longer periods of time if convenient or desired, in some embodiments, this step is carried out for less than about 5 minutes, (e.g., less than about 5 minutes, less than about 4 minutes, less than about 3 minutes, less than about 2 minutes, or less than about 1 minute), as this time period is sufficient to allow for efficient release of the bound nucleic acids. In some embodiments, the step of contacting the nucleic acid bound polymer or composition with the elution solution is conducted for about 10 seconds to about 10 minutes, or about 1 minute to about 5 minutes.

In embodiments in which an elution solution is used, no chaotropic agent (e.g., guanidine hydrochloride or guanidine isothiocyanate), organic solvent (e.g., alcohols such as ethanol or methanol, or other organic solvents such as dimethylsulfoxide), or detergent (e.g., sodium dodecyl sulfate) in the elution solution. Even trace amounts of such components in a solution of target nucleic acid can severely limit the utility of the nucleic acid in downstream processing or analysis. This is another significant advantage of disclosed polymers or compositions—in embodiments, the nucleic acids can be rapidly released from the polymers or compositions using aqueous buffer solutions that do not include components that would interfere with downstream applications.

When particular pH conditions are required in any of the method steps (e.g., the contacting step, optional washing steps, and the elution step), the pH of the sample or solution can be adjusted to the indicated pH value using an acidifying agent or a basifying agent, as required. Suitable acidifying agents include, but are not limited to: inorganic acids such as HCl, HBr, HClO₄, HClO₃, H₂SO₄, H₂SO₃, H₃PO₄, or H₃PO₃; organic acids such as 2-acetyoxybenzoic, acetic, ascorbic, aspartic, benzoic, camphorsulfonic, cinnamic, citric, edetic, ethanedisulfonic, ethanesulfonic, fumaric, glucoheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, tetrafluoroboric, toluenesulfonic, trifluoroacetic, trifluoromethanesulfonic, and valeric acids. Suitable basifying agents include but are not limited to inorganic bases such as sodium hydroxide, potassium hydroxide, calcium hydroxide, sodium bicarbonate, sodium carbonate, ammonia, diethanolamine, meglumine, lysine, arginine, ethanolamine, piperazine, trometamol, triethanolamine, and the like.

In some embodiments, the nucleic acid bound polymer or composition can be used directly in a downstream application without an elution step. For example, in some embodiments, the nucleic acid bound polymer or composition can be used directly in an on-bead polymerase chain reaction.

In some embodiments, the isolated nucleic acid can be used in any number of downstream applications, such as methods that analyze or further process the nucleic acid. For example, in some embodiments, the disclosed methods further comprise a step of analyzing or processing the nucleic acid, e.g., using polymerase chain reaction (PCR), reverse transcription PCR (RT-PCR), quantitative PCR, quantitative reverse transcription PCR (RT-qPCR), real time PCR, hot start PCR, single cell PCR, nested PCR, in situ colony PCR, digital PCR (dPCR), Droplet Digital™ PCR (ddPCR), emulsion PCR, ligase chain reaction (LCR), transcription based amplification system (TAS), nucleic acid sequence-based amplification (NASBA), strand displacement amplification (SDA), rolling circle amplification (RCA), hyper-branched RCA (HRCA), isothermal amplification, gel electrophoresis, capillary electrophoresis, mass spectrometry, fluorescence detection, ultraviolet spectrometry, hybridization assays, DNA or RNA sequencing, reverse transcription, next generation sequencing (NGS), or the like.

### IV. Examples

### Example 1

### Synthesis of Polymer-1

1 g of morpholine acrylate monomer (e.g., 2-(3-(3-morpholinopropyl)ureido)ethyl methacrylate) and 83 mg of 3-(trimethoxysilyl)propyl methacrylate were dissolved in 20 ml of anhydrous THF. The solution was degassed with N₂ for 30 min. 27 mg of AIBN (azobisisobutyronitrile, an initiator) was added, and the solution was heated up to 65° C. for 4 hours. The polymer was precipitated out using 10× diethyl ether followed by washing with 10× EtOH. The precipitation and EtOH washing was repeated 3 times to remove any side products. The polymer was then stored in 5 ml EtOH.

### Example 2

### Synthesis of Polymer-2

1 g of morpholine acrylate monomer (e.g., 2-(3-(3-morpholinopropyl)ureido)ethyl methacrylate) was dissolved in 20 ml of anhydrous THF. The solution was degassed with N₂ for 30 min. 27 mg of AIBN (an initiator) was added, and the solution was heated up to 65° C. for 4 hours. The polymer was precipitated out using 10× diethyl ether followed by washing with 10× EtOH. The precipitation and EtOH washing was repeated 3 times to remove any side products. The polymer was then stored in 5 ml EtOH.

### Example 3

### Preparation of Polymer-Functionalized Magnetic Silica Beads (1936-1)

5 ml of Polymer-1 (70 mg/ml) in EtOH was added into 20 ml of 0.9M ammonium sulfate aqueous solution. 0.5 g of magnetic silica beads was then added into the polymer solution. The solution was mixed for overnight. The covalently functionalized magnetic silica beads (1936-1) were washed with water 5 times and then stored in water.

### Example 4

### Preparation of Polymer-Functionalized Magnetic Silica Beads (1936-2)

5 ml of Polymer-2 (70 mg/ml) in EtOH was added into 20 ml of 0.9M ammonium sulfate aqueous solution. 0.5 g of magnetic silica beads was then added into the polymer solution. The solution was mixed for overnight. The noncovalently functionalized magnetic silica beads (1936-2) were washed with water 5 times and then stored in water.

### Example 5

### DNA Binding and Elution Assay

The two sets of functionalized/coated magnetic silica beads prepared in Example 3 and Example 4 were tested for binding and elution of nucleic acids from a sample: 1 mg of resins was added into 100 ng/ml DNA in 10 mM PBS 7.4 and 200 mM NaCl and mixed for 1 min. Resins were removed, and nucleic acid in the supernatant was quantified to calculate the binding efficiency. DNA in the resins was then eluted out in 50 mM Tris pH 9 for 1 min and quantified by Quantifluor dsDNA fluorescent dye to calculate the elution efficiency.

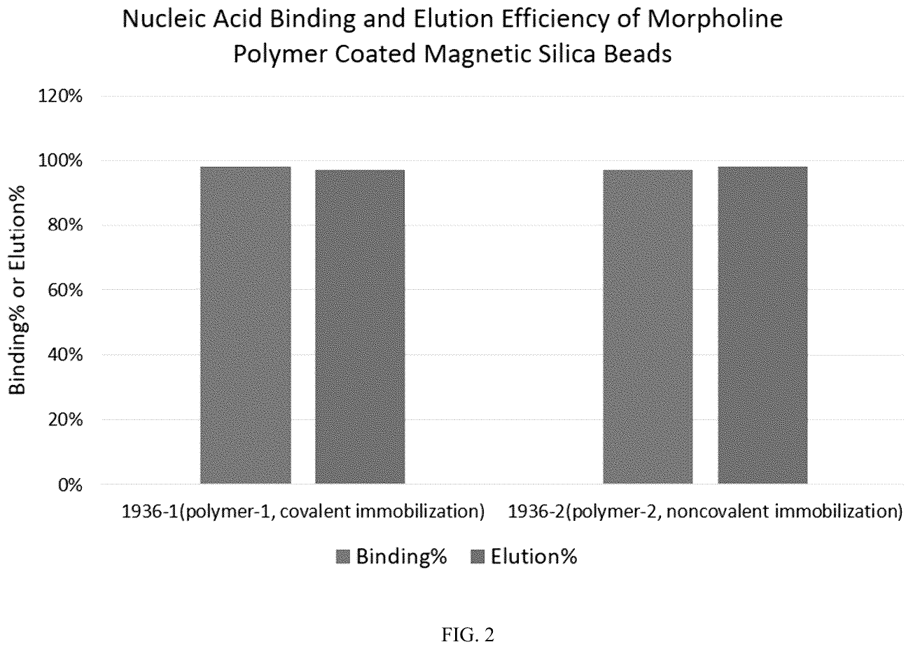

Results are shown in FIG. 2. The data demonstrate that both beads can bind and elute DNA rapidly and quantitatively.

### Example 6

### DNA Binding and Elution Assay

The two sets of functionalized/coated magnetic silica beads prepared in Example 3 were used to isolate circulating cell-free DNA from human blood plasma. 1 mg of beads was incubated with plasma in a low pH buffer (<6.5), washed, and then eluted with a higher pH buffer (>9.0). A Promega Maxwell Instrument was used to automate the process. The nucleic acid concentration of the eluates was then quantified using qPCR.

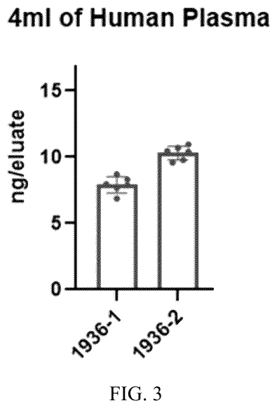

Results are shown in FIG. 3 as total ng of ccfDNA isolated per eluate. Both functionalized/coated beads were able to isolate amplifiable ccfDNA from human plasma. Isolation of ccfDNA from 4 mL of human plasma was performed in triplicate and quantification by qPCR was conducted in duplicate for each eluate for a total of six data points, each represented in FIG. 3.

1. A composition comprising:
a solid surface; and
a polymer covalently or non-covalently bound to the solid surface;
wherein the polymer comprises at least one pendant moiety of formula (I): 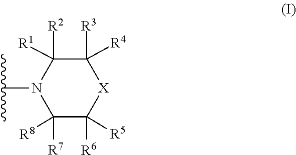
wherein:
X is selected from O, S, S(O), and S(O)₂; and
R¹, R², R³, R⁴, R⁵, R⁶, R⁷, and R⁸ are each independently selected from hydrogen, C₁-C₆ alkyl, C₂-C₆ alkenyl, C₂-C₆ alkynyl, C₁-C₆ haloalkyl, C₃-C₆ cycloalkyl, aryl, heteroaryl, heterocyclyl, halo, cyano, nitro, —OR^{a}, —SR^{b}, —C(O)OR^{c}, —C(O)NR^{d}R^{e}, —OC(O)NR^{f}R^{g}, —NR^{h}C(O)NRⁱR^{j}, and —NR^{h}C(S)NRⁱR^{j}, wherein each R^{a}, R^{b}, R^{c}, R^{d}, R^{e}, R^{f}, R^{g}, R^{h}, Rⁱ, and R^{j} is independently selected from hydrogen, C₁-C₆ alkyl, C₂-C₆ alkenyl, C₂-C₆ alkynyl, C₁-C₆ haloalkyl, C₃-C₆ cycloalkyl, and aryl.

2. The composition of claim 1, wherein the solid surface comprises a material selected from silica, glass, a polymer, and a metal.

3. The composition of claim 1, wherein the solid surface comprises a polymer selected from cellulose, cellulose acetate, nitrocellulose, nylon, a polyester, a polyethylene, a polyethersulfone, a polyolefin, polyvinylidene fluoride, a polyacrylate, a polystyrene, or any combination thereof.

4. The composition of claim 1, wherein the solid surface is in the form of a bead, a resin, a magnetic particle, a membrane, a vial, a plate, a film, a tube, a syringe, a cartridge, a cassette, a pipette tip, a microfluidic cartridge, or a cuvette.

5. (canceled)

6. The composition of 1 wherein the polymer comprises a backbone and a plurality of pendant moieties of formula (I), wherein each pendant moiety of formula (I) is bound to the backbone via a linker comprising one or more —CH₂—, —O—, —C(O)O—, —C(O)NH—, —NHC(O)O—, —OC(O)O—, —NHC(O)NH—, —S—, —C(O)S—, —C(S)NH—, —NHC(S)O—, —OC(S)O—, —NHC(S)NH—, arylene, heteroarylene, or heterocyclylene moieties, or any combination thereof.

7. The composition of claim 6, wherein the linker has a structure:
—(CH₂)ₙ₁—Z¹—(CH₂)ₙ₂—Z²—
wherein n1 and n2 are each independently selected from 1, 2, 3, 4, 5, 6, 7, and 8, and Z¹ and Z² are each independently selected from —NHC(O)NH—, —C(O)NH—, —OC(O)NH—, —OC(O)O—, —NHC(O)S—, —NHC(S)NH—, —O—, —C(O)O—, and a bond.

8. The composition of claim 6, wherein the linker is selected from:

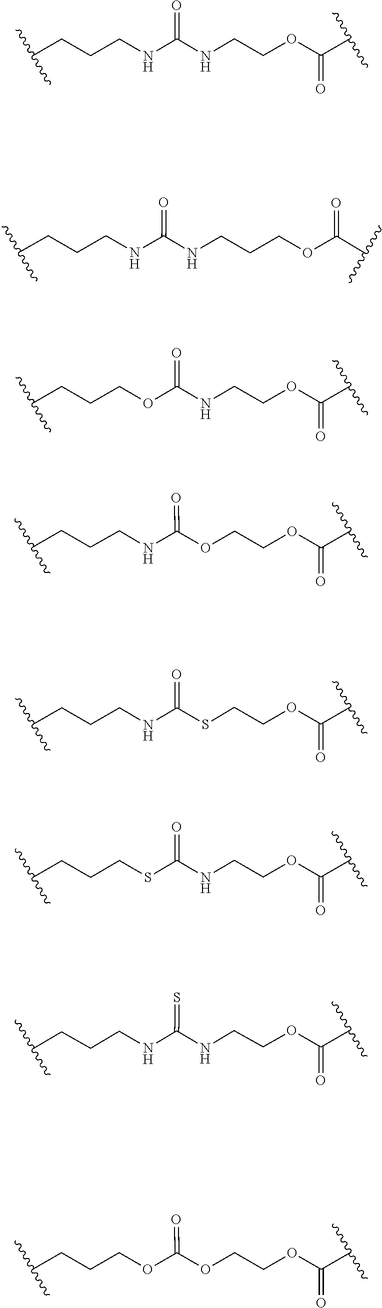

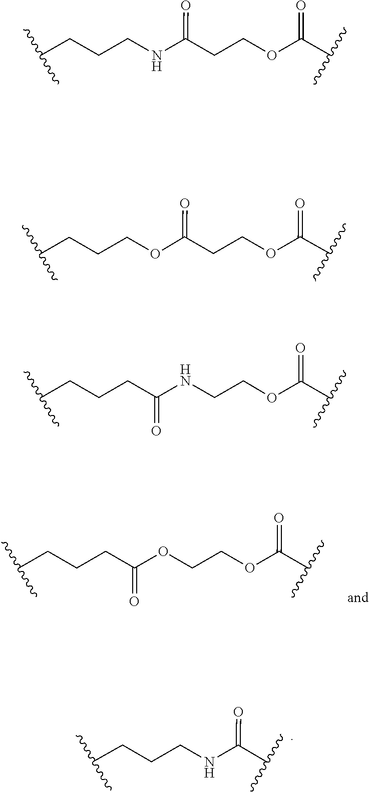 [SMILES: *CCCNC(=O)CCOC(*)=O ; *CCCOC(=O)CCOC(*)=O ; *CCCC(=O)NCCOC(*)=O ; *CCCC(=O)OCCOC(*)=O ; *CCCNC(*)=O]

9. (canceled)

10. The composition of claim 1, wherein X is O, and wherein R¹, R², R³, R⁴, R⁵, R⁶, R⁷, and R⁸ are each hydrogen.

11. (canceled)

12. The composition of claim 1, wherein the polymer comprises a monomer unit of formula (Ia): 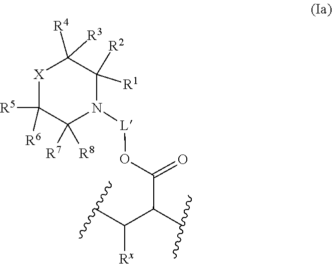
wherein:
L′ is a linker of formula: —(CH₂)ₙ₁—Z¹—(CH₂)ₙ₂—Z²—, wherein n1 and n2 are each independently selected from 1, 2, 3, 4, 5, 6, 7, and 8, and Zⁱ and Z² are each independently selected from —NHC(O)NH—, —C(O)NH—, —OC(O)NH—, —OC(O)O—, —NHC(O)S—, —NHC(S)NH—, —O—, —C(O)O—, and a bond;
Q is O or NH; and
R^{x} is selected from hydrogen and methyl.

13. The composition of claim 12, wherein the monomer unit of formula (Ia) is a monomer unit of formula:

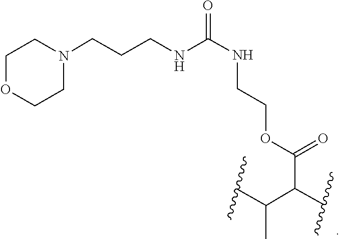

14. The composition of claim 1, wherein the polymer further comprises one or more (meth)acrylate or (meth)acrylamide monomer units, wherein at least one (meth)acrylate or (meth)acrylamide monomer unit comprising a trialkoxysilyl group.

15. (canceled)

16. The composition of claim 14, wherein the polymer further comprises at least one monomer unit having the formula: 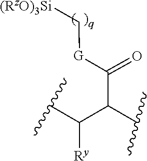
wherein:
R^{y} is selected from hydrogen and methyl;
each R^{z} is independently C₁-C₄ alkyl;
G is O or NH; and
q is 1, 2, 3, 4, 5, or 6.

17. The composition of claim 16, wherein each R^{z} is methyl, G is O, and q is 3.

18. The composition of claim 1, wherein the polymer is covalently bound to the solid surface.

19. The composition of claim 1, wherein the polymer is non-covalently bound to the solid surface.

20. The composition of claim 1, further comprising a second polymer covalently or non-covalently bound to the solid surface, wherein the second polymer comprises at least one pendant moiety of formula (I).

21. (canceled)

22. The composition of claim 1, wherein the solid surface further comprises at least one polyethylene glycol (PEG) moiety bound to the surface.

23. A method of isolating a nucleic acid from a sample, comprising:
(a) providing a sample containing a nucleic acid;
(b) contacting the sample with a composition of claim 1 at a pH of less than about 8.0, wherein the nucleic acid binds to the composition to provide a nucleic acid bound composition; and
(c) separating the nucleic acid bound composition from the sample.

24.-51. (canceled)

52. A method of isolating a nucleic acid from a sample, comprising:
(a) providing a sample containing a nucleic acid;
(b) contacting the sample with a polymer at a pH of less than about 8.0, wherein the nucleic acid binds to the polymer to provide a nucleic acid bound polymer; and
(c) separating the nucleic acid bound polymer from the sample,
wherein the polymer comprises at least one pendant moiety of formula (I): 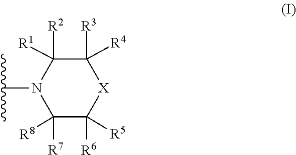
wherein:
X is selected from O, S, S(O), and S(O)₂; and
R¹, R², R³, R⁴, R⁵, R⁶, R⁷, and R⁸ are each independently selected from hydrogen, C₁-C₆ alkyl, C₂-C₆ alkenyl, C₂-C₆ alkynyl, C₁-C₆ haloalkyl, C₃-C₆ cycloalkyl, aryl, heteroaryl, heterocyclyl, halo, cyano, nitro, —OR^{a}, —SR^{b}, —C(O)OR^{c}, —C(O)NR^{d}R^{e}, —OC(O)NR^{f}R^{g}, —NR^{h}C(O)NRⁱR^{j}, and —NR^{h}C(S)NRⁱR^{j}, wherein each R^{a}, R^{b}, R^{c}, R^{d}, R^{e}, R^{f}, R^{g}, R^{h}, Rⁱ, and R^{j} is independently selected from hydrogen, C₁-C₆ alkyl, C₂-C₆ alkenyl, C₂-C₆ alkynyl, C₁-C₆ haloalkyl, C₃-C₆ cycloalkyl, and aryl.

53.-93. (canceled)

94. A copolymer comprising:
(a) a plurality of monomer units of formula (Ia): 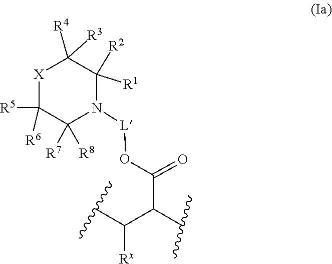
wherein:
X is selected from O, S, S(O), and S(O)₂;
R¹, R², R³, R⁴, R⁵, R⁶, R⁷, and R⁸ are each independently selected from hydrogen, C₁-C₆ alkyl, C₂-C₆ alkenyl, C₂-C₆ alkynyl, C₁-C₆ haloalkyl, C₃-C₆ cycloalkyl, aryl, heteroaryl, heterocyclyl, halo, cyano, nitro, —OR^{a}, —SR^{b}, —C(O)OR^{c}, —C(O)NR^{d}R^{e}, —OC(O)NR^{f}R^{g}, NR^{h}C(O)NRⁱR^{j}, and —NR^{h}C(S)NRⁱR^{j} wherein each R^{a}, R^{b}, R^{c}, R^{d}, R^{e}, R^{f}, R^{g}, R^{h}, Rⁱ, and R^{j} is independently selected from hydrogen, C₁-C₆ alkyl, C₂-C₆ alkenyl, C₂-C₆ alkynyl, C₁-C₆ haloalkyl, C₃-C₆ cycloalkyl, and aryl,
L′ is a linker of formula: —(CH₂)ₙ₁—Z¹—(CH₂)ₙ₂—Z²—, wherein n1 and n2 are each independently selected from 1, 2, 3, 4, 5, 6, 7, and 8, and Z¹ and Z² are each independently selected from —NHC(O)NH—, —C(O)NH—, —OC(O)NH—, —OC(O)O—, —NHC(O)S—, —NHC(S)NH—, —O—, —C(O)O—, and a bond;
Q is O or NH; and
R^{x} is selected from hydrogen and methyl; and
(b) a plurality of monomer units of formula (Ib): 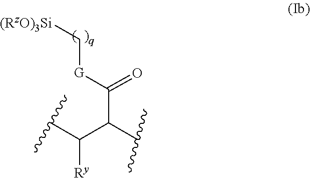
wherein:
R^{y} is selected from hydrogen and methyl;
each R^{z} is independently C₁-C₄ alkyl;
G is O or NH; and
q is 1, 2, 3, 4, 5, or 6.
